(19) United States
(12) Patent Application Publication
(10) Pub. No.: US 2003/0082170 A1
(43) Pub. Date: May 1, 2003

(54) Protein from brucella species

(76) Inventors: Luther E. Lindler, Wheaton, MD (US); Richard Warren, Blue Bell, PA (US); Lillian VanDeVerg, Gaithersburg, MD (US); Fran Rubin, Bethesda, MD (US)

Correspondence Address: Elizabeth Arwine, Patent Attorney, U.S. Army Medical Research & Materiel Command, 504 Scott Street, Fort Detrick, MD, 21702-5012, US

(21) Appl. No.: 09/785,689

(22) Filed: Feb. 20, 2001

Related U.S. Application Data
(63) Continuation of application No. 09/082,535, filed on May 21, 1998, now abandoned.
Application No. 09/082,535 is a continuation-in-part of application No. 08/446,103, filed on May 19, 1995, now abandoned.

(51) Int. Cl. A01N 37/18; A61K 38/00; C12P 21/04; A61K 39/395; A61K 39/02; C07K 1/00; C07K 14/00; C07K 17/00; C07K 16/00

(52) U.S. Cl. 424/130.1; 530/350; 514/2; 435/70.21

(57) ABSTRACT

A 28 kDa protein which has use as a diagnostic agent for identifying antibodies to Brucella species and as vaccines to raise antibodies against several species of Brucella has been purified.

### APPLICATION FOR UNITED STATES PATENT

This application is a continuation-in-part of U.S. Ser. No. 08/446,103.

### FIELD OF THE INVENTION

This invention relates to a 28 kDa protein which has use as a diagnostic agent for identifying antibodies to Brucella species and as vaccines to raise antibodies against Brucella.

### BACKGROUND OF THE INVENTION

Brucellosis is a disease caused by microorganisms of the genus Brucella. B. abortus infects cattle, causing abortion. B. melitensis infects sheep and goats. B. ovis infects birds and B. canis infects dogs. Infection usually results from contact with secretions and/or excretions from infected animals. At one time, infection was usually the result of ingestion of milk or products of milk from infected animals. The disease is rarely transmitted from person to person. Persons more frequently exposed to the disease include meat packers, veterinarians, farmers and livestock producers. Raw milk, which was once a common source of infection in North America, no longer presents a serious problem, since commercially available milk is pasteurized. However, milk and milk products still present a source of infection in countries where pasteurization is not practiced routinely.

In humans, the onset of disease may be accompanied by chills, fever, headache and muscular pain. At other times, the onset is insidious. The patient may suffer mild malaise, muscular, head and neck pain. The disease may result in temperatures of 40° C. to 41° C. In some cases, the patient may suffer intermittent recurrence over a period of months or years. Because the disease can mimic many other disease conditions, an inexpensive, reliable diagnostic test for the disease is needed.

All of the current diagnostic tests for brucellosis rely on agglutination of killed whole cells or immunodiffusion of whole cell extracts. The Brucella preparations are made from smooth bacteria i.e., the bacteria used in these tests have full length lipopolysaccharide (LPS). During infections with smooth species or strains of BRUCELLA, the vast majority of the antibody response is directed against the LPS of the organism. This presents two problems for the test diagnosis of brucellosis: 1) Animals, including humans, which are infected with rough species (lacking full length LPS) of Brucella such as B. ovis or B. canis, will test negative for infection with these tests. Furthermore, cross-reactivity between Brucella LPS and LPS from other bacteria results in false positives. 2) Animals that have been vaccinated with one of the live attenuated strains of B. abortus but do not have disease will test positive with the prior art tests due to the production of antibodies against the LPS of the vaccine strain. This second problem is currently not an issue with humans, because there are no live vaccine strains which are not pathogenic to humans.

At present, attempts are being made to find a protein-based test for brucellosis. A test of this nature would solve the problems associated with the prior art methods. Currently, the meat packing industry does not test animals before slaughter because of their inability to differentiate between vaccinated and diseased animals.

Two papers in the literature describe antibody recognition of Brucella proteins. Goldbaum, et al., (J. Clin. Microbiol 31:2141-2145 (1993)) has described an 18 kDa protein which is recognized by human convalescent serum. A 20 kDa protein that was discovered by Zygmunt, et al. (J. Clin Microbiol 30:2662-2667 (1992)) is recognized by serum obtained from infected sheep. All of the other immunoreactive proteins described for Brucella species have been shown to be heat shock proteins which are ubiquitous in nature and highly conserved. Accordingly, these other proteins are of no value in diagnostic tests and may cause complications if used in vaccines.

In 1995, Debbarh, et al. reported an intracellular protein having a molecular weight of about 28 kDa. ( Veterinary Microbiology 44, (1995) pp 37-48.)

Riezu-Boj, et al. (Infection and Immunity, (February 1990), pp. 489-494) have identified several outer membrane proteins. The sera of that reference has been tested. It has been found that the instantly claimed protein does not react with that sera.

### DESCRIPTION OF THE INVENTION

The instant invention is an antigenic membrane protein of about 28 kDa which has been characterized and produced by recombinant technology. The protein interacts with antibodies against a variety of Brucella species. The inventive protein is effective at eliciting immune response.

The protein described herein measures antibodies against proteins of the organism. Diagnostic probes based on the nucleotide sequence of the 28 kDa antigen can be made.

Finally, since the protein gives rise to immune response, the protein may be used as a submit vaccine for brucellosis. Compositions containing the subject proteins or polypeptides of the protein in pharmaceutically acceptable carriers are useful as vaccines and as diagnostic agents to identify protective antibodies. The proteins may be attached to solid supports for use in diagnostic studies known in the art.

Both the 28 kDA protein and polypeptides containing over 90 amino acid peptides of the protein thereof can be may be administered by mouth. Antigenic fusion proteins containing sequences of other proteins such as cholera subunits are useful for administration orally or to the mucosa (for example intra-nasally). The fusion proteins may be lyophilized and inhaled from a vial for administration.

Materials and Methods:

A genomic library of B. melitensis DNA was generated in lambda gt11 obtained from Promega Corp., Madison, Wis. The bacterial genomic DNA was digested with HaeIII to generate fragments with an average size of 2.5 kilo base-pairs (kb). The DNA was then methylated with EcoRI methylase followed by addition of EcoRI linkers and digestion with EcoRI restriction enzyme. The prepared fragments then were added to EcoRI cleaved lambda gt11 arms, ligated and packaged. The library was judged to be complete by determining the average insert size for a group of 20 random plaques and using the method of Sambrook, et al. (Molecular Cloning: A Laboratory Manual, Col Spring Harbor Press, Cold Spring Harbor Press, Cold Spring Harbor, N.Y. (1989)).

Antibodies were purified from brucellosis patients in Egypt and were used to screen the library for recombinant phage that encoded immunoreactive B. melitensis proteins. Immunoreactive plaques were purified and characterized further. The isolate described herein was designated clone “4D”. To determine which Brucella antigen 4D encoded, antibody was extracted from a plaque lift of pure phage and used to probe a western blot of whole cell extracts prepared from B. melitensis RM1., B.abortus RA1 and a negative control, E coli Y1090. It was revealed that 4D encoded a 28 kilodalton (kDa) antigen.

The genomic DNA was extracted from the recombinant virus for cloning into a plasmid vector to facilitate characterization of the gene. During this process, it was found that one of the EcoRI recognition sites was not present. This site was the one “downstream” in relation to the vector lacZ gene. As a result, Brucella DNA contained in 4D was cloned on an approximately 5 kb EcoRI to KpnI fragment into vector pSK obtained from Stratagene Corporation, La Jolla, Calif.

Several sub-clones of the original pBM4D1 were made and the extracts were subjected to analysis by western blot. The DNA sequence of interest shown below:

cccctgacataacccgctttgtccaaattttttcaacttttcctgtagga
gattttatgaacactcgtgctagcaattttctcgcagcctcattttccac
aatcatgctcgtcggcgctttcagcctgcccgctttcgcacaggagaatc
agatgacgacgcagcccgcgcgcatcgccgtcaccggggaaggcatgatg
acggcctcgcccgatatggccattctcaatctctcggtgctacgccaggc
aaagaccgcgcgcgaagccatgaccgcgaataatgaagccatgacaaaag
tgctcgatgccatgaagaaggccggcatcgaagatcgcgatctccagaca
ggcggcatcaatatccagccgatttatgtctatcctgacgacaagaacaa
cctgaaagagcctaccatcaccggctattctgtatccaccagtctcacgg
ttcgcgtgcgcgaactggccaatgttggaaaaattttggatgaatccgtc
acgctcggtgttaatcagggcggtgatttgaacctggtcaatgataatcc
ctccgccgtgatcaacgaggcgcgcaagcgcgcagtggccaatgccattg
ccaaggcgaagacgcttgccgacgctgcaggcgtggggcttggccgtgtg
gtggaaatcagtgaactgagccgcccgcccatgccgatgccaattgcgcg
cggacagttcagaaccatgctagcagccgcaccggacaattccgtgccga
ttgccgcaggcgaaaacagctataacgtatcggtcaatgtcgtttttgaa
atcaagtaaatagctggggtatgacgccctttgccacctgatacaaaacg
ccggcctggtttcacaggccggtttttttgattagagcgcgtttcgatct
gattgaatccgatcggcgctctaatcctttgttttgacgcgcatcttttc
cgaaaaccgtttcacacttttcgggatgcggtctagcggatgatcgggca
accgcgcgtatcggcaaatgtcacg

A database search of the National Center for Biotechnology Information database was performed. However, no significant homologies were found. The gene encodes the 28 kDa antigen contained within the genomic material of Brucella species. The DNA/amino acid sequence was identified to be:

| | |
|---|---|
| 1 | |
| cccctgacataacccgctttgtccaaattttttcaacttttcctg | 60 |
| 1 | 20 |
| taggagattttatga | |
| M N | |
| acactcgtgctagcaattttctcgcagcctcattttccacaatca | 120 |
| T R A S N F L A A S F S T I M | 40 |
| tgctcgtcggcgctt | |
| L V G A F | |
| tcagcctgcccgctttcgcacaggagaatcagatgacgacgcagc | 180 |
| S L P A F A Q E N Q M T T Q P | 60 |
| ccgcgcgcatcgccg | |
| A R I A V | |
| tcaccggggaaggcatgatgacggcctcgcccgatatggccattc | 240 |
| T G E G M M T A S P D M A I L | 80 |
| tcaatctctcggtgc | |
| N L S V L | |
| tacgccaggcaaagaccgcgcgcgaagccatgaccgcgaataatg | 300 |
| R Q A K T A R E A M T A N N E | 100 |
| aagccatgacaaaag | |
| A M T K V | |
| tgctcgatgccatgaagaaggccggcatcgaagatcgcgatctcc | 360 |
| L D A M K K A G I E D R D L Q | 120 |
| agacaggcggcatca | |
| T G G I N | |
| atatccagccgatttatgtctatcctgacgacaagaacaacctga | 420 |
| I Q P I Y V Y P D D K N N L K | 140 |
| aagagcctaccatca | |
| E P T I T | |
| ccggctattctgtatccaccagtctcacggttcgcgtgcgcgaac | 480 |
| G Y S V S T S L T V R V R E L | 160 |
| tggccaatgttggaa | |
| A N V G K | |
| aaattttggatgaatccgtcacgctcggtgttaatcagggcggtg | 540 |
| I L D E S V T L G V N Q G G D | 180 |
| atttgaacctggtca | |
| L N L V N | |
| atgataatccctccgccgtgatcaacgaggcgcgcaagcgcgcag | 600 |
| D N P S A V I N E A R K R A V | 200 |
| tggccaatgccattg | |
| A N A I A | |
| ccaaggcgaagacgcttgccgacgctgcaggcgtggggcttggcc | 660 |
| K A K T L A D A A G V G L G R | 220 |
| gtgtggtggaaatca | |
| V V E I S | |
| gtgaactgagccgcccgcccatgccgatgccaattgcgcgcggac | 720 |
| E L S R P P M P M P I A R G Q | 240 |
| agttcagaaccatgc | |
| F R T M L | |
| tagcagccgcaccggacaattccgtgccgattgccgcaggcgaaa | 780 |
| A A A P D N S V P I A A G E N | 260 |
| acagctataacgtat | |
| S Y N V S | |
| cggtcaatgtcgtttttgaaatcaagtaaatagctggggtatgac | 840 |
| V N V V F E I K * | 280 |
| gccctttgccacctg | |
| atacaaaacgccggcctggtttcacaggccggtttttttgattag | 900 |
| agcgcgtttcgatct | |
| gattgaatccgatcggcgctctaatcctttgttttgacgcgcatc | 960 |
| ttttccgaaaaccgt | |
| ttcacacttttcgggatgcggtctagcggatgatcgggcaaccgc | 1020 |
| gcgtatcggcaaatg | |
| tcacg 1025 | |

Southern blots with a 28 kDa specific probe revealed that the gene is contained within a 8.5 kb EcoRI fragment in B. melitensis 16 M and Rev1, B. abortus 2308 and S19, B. suis, B. neotome, B. Canis and B. ovis.

The 28 kDa Brucella protein was shown to have the sequence:

Met Asn Thr Arg Ala Ser Asn Phe Leu Ala Ala Ser
Phe Ser Thr Ile Met Leu Val Gly Ala Phe Ser Leu
Pro Ala Phe Ala Gln Glu Asn Gln Met Thr Thr Gln
Pro Ala Arg Ile Ala Val Thr Gly Glu Gly Met Met
Thr Ala Ser Pro Asp Met Ala Ile Leu Asn Leu Ser
Val Leu Arg Gln Ala Lys Thr Ala Arg Glu Ala Met
Thr Ala Asn Asn Glu Ala Met Thr Lys Val Leu Asp
Ala Met Lys Lys Ala Gly Ile Glu Asp Arg Asp Leu
Gln Thr Gly Gly Ile Asn Ile Gln Pro Ile Tyr Val
Tyr Pro Asp Asp Lys Asn Asn Leu Lys Glu Pro Thr
Ile Thr Gly Tyr Ser Val Ser Thr Ser Leu Thr Val
Arg Val Arg Glu Leu Ala Asn Val Gly Lys Ile Leu
Asp Glu Ser Val Thr Leu Gly Val Asn Gln Gly Gly
Asp Leu Asn Leu Val Asn Asp Asn Pro Ser Ala Val
Ile Asn Glu Ala Arg Lys Arg Ala Val Ala Asn Ala
Ile Ala Lys Ala Lys Thr Leu Ala Asp Ala Ala Gly
Val Gly Leu Gly Arg Val Val Glu Ile Ser Glu Leu
Ser Arg Pro Pro Met Pro Met Pro Ile Ala Arg Gly
Gln Phe Arg Thr Met Leu Ala Ala Ala Pro Asp Asn
Ser Val Pro Ile Ala Ala Gly Glu Asn Ser Tyr Asn
Val Ser Val Asn Val Val Phe Glu Ile Lys

The 28 kDa protein is obtained from lysates of the cell cultures and is purified by ion exchange to apparent homogeneity. Other methods known in the art appropriate for use include reverse phase HPLC and size exclusion chromatography.

Western blots of whole cell extracts of E. coli containing either pBM4D1 or pSK negative control were studied. All primary antisera were diluted 1:1,000 relative to their original concentration before use. The results are shown in Table I. The proteins of the invention may also be used in other testing methods such as immunofluorescence and ELISA tests.

Comparison of Protein of the Invention with Sera Utilized in Riezu-Boj:

The protein of this invention was exposed to sera used in Riezu-Boj to determine whether the protein of the invention was reactive therewith. The protein identified as Omp28 (protein of this invention) and proteins of Riezu-Boj were identified on a Western blot. Antibodies against the Omp28 of the invention were then applied to the Western blot. The antibodies raised to the protein Omp28 of the invention interacted with Omp28 protein, but did not react with any protein from the Riezu-Boj sera. Hence, it was shown that the proteins of Riezu-Boj has different immunological properties from the protein of the invention.

Protection against brucellosis using the 28 kDa protein as a vaccine requires administration of a composition containing an immunogenic effective amount of the 28 kDA protein in a pharmaceutically acceptable carrier. Such compositions may be administered to the mucosa or by mouth. The compositions may also be administered parenterally. Preferred parenteral routes include intracutaneous or subcutaneous or intramuscular injection. Any of the compositions may contain, additionally, adjuvants such as alum or Freund's adjuvant.

TABLE 1
ANTIBODY REACTIVITY WITH 28 kDa PROTEIN

| Sample | IgM | IgG |
|---|---|---|
| Human | | |
| 7445 | + | + |
| 7468 | + | + |
| 8093 | − | + |
| 8150 | + | + |
| 8160 | − | + |
| 8230 (8089)_{b} | − | − |
| 8755 | + | + |
| 8855 | + | − |
| CG | + | − |
| Mouse | ND_{c} | + |
| Rabbit | ND | + |

ₐResults of western blots of whole cell extracts of E. coli containing either pBM4D1 or pSK negative control. All primary antisera were diluted 1:1000 of their original concentration before use.
_{b}Sample 8230 was positive for IgM anti-28 kDa at a primary antibody dilution of 1:250.
_{c}ND = not determined

Hybrid gene fusion proteins may also be produced to increase protective immune response. For example, DNA sequences which encode the 28 kDa protein or antigenic polypeptides therefrom may be fused to DNA sequences which encode non-toxic peptides of other organisms such as cholera. U.S. Pat. No. 5,268,276 to Holmgren et al., which is incorporated herein in its entirety by reference, discloses a means of producing appropriate fusion gene to produce fusion proteins containing he 28 kDa protein or the immunogenic peptides therefrom.

Immunogens are prepared by exposing the lysate to gel electrophoresis, then excising the 28 kDa bands from gel. The 28 kDa-containing band is then subjected to electrophoretic purification. The bands are pulverized in liquid nitrogen, then mixed with complete adjuvant and injected subcutaneously into the rabbits. The animals are given boosters after two weeks. Three to four days after the booster injection, the sera containing polyclonal antibodies is collected and screened. Serum containing antibodies can be detected by Western-blot.

Antibodies prepared against the 28 kDa antigen may be used to identify the infectious organisms in body fluids of mammals suspected of having brucellosis. Activity of monoclonal and polyclonal antibodies against 28 kDa antigens can be tested by several means. Western blot is used for the initial screening using 28 kDa bands as electrophoretically transferred to nitrocellulose paper. This screening procedure selects for high affinity antibodies, since they must survive stringent washing methods. The monoclonal antibodies are used in screening the cDNA libraries. ELISA methodology can also be used as an alternate for initial screening.

1. a 28 kda protein which is reactive with antibodies raised to brucella species have a sequence of at least 90 amino acids from the sequence:
Met Asn Thr Arg Ala Ser Asn Phe Leu Ala Ala Ser
Phe Ser Thr Ile Met Leu Val Gly Ala Phe Ser Leu
Pro Ala Phe Ala Gln Glu Asn Gln Met Thr Thr Gln
Pro Ala Arg Ile Ala Val Thr Gly Glu Gly Met Met
Thr Ala Ser Pro Asp Met Ala Ile Leu Asn Leu Ser
Val Leu Arg Gln Ala Lys Thr Ala Arg Glu Ala Met
Thr Ala Asn Asn Glu Ala Met Thr Lys Val Leu Asp
Ala Met Lys Lys Ala Gly Ile Glu Asp Arg Asp Leu
Gln Thr Gly Gly Ile Asn Ile Gln Pro Ile Tyr Val
Tyr Pro Asp Asp Lys Asn Asn Leu Lys Glu Pro Thr
Ile Thr Gly Tyr Ser Val Ser Thr Ser Leu Thr Val
Arg Val Arg Glu Leu Ala Asn Val Gly Lys Ile Leu
Asp Glu Ser Val Thr Leu Gly Val Asn Gln Gly Gly
Asp Leu Asn Leu Val Asn Asp Asn Pro Ser Ala Val
Ile Asn Glu Ala Arg Lys Arg Ala Val Ala Asn Ala
Ile Ala Lys Ala Lys Thr Leu Ala Asp Ala Ala Gly
Val Gly Leu Gly Arg Val Val Glu Ile Ser Glu Leu
Ser Arg Pro Pro Met Pro Met Pro Ile Ala Arg Gly
Gln Phe Arg Thr Met Leu Ala Ala Ala Pro Asp Asn
Ser Val Pro Ile Ala Ala Gly Glu Asn Ser Tyr Asn
Val Ser Val Asn Val Val Phe Glu Ile Lys.

2. A composition of matter comprising protein of claim 1 attached to a support.

3. A composition of matter comprising a protein of claim 1 attached to an antibody.

4. A composition of matter comprising a protein of claim 1 and a pharmaceutically acceptable carrier.

5. A composition of matter of claim 3 wherein the antibody is attached to a solid support.

6. A composition of claim 5 wherein the support is a nitro-cellulose strip.

7. An antibody which reacts selectively with the protein of claim 1.

8. A composition of matter comprising at least one antibody of claim 7 in a carrier.

9. A composition of claim 4 containing, additionally, an adjuvant.

10. A method of raising antibodies against Brucella species in a mammal susceptible to brucellosis by administration of an immunogenic effective amount of a protein of claim 1.
